(19) United States
(12) Patent Application Publication
(10) Pub. No.: US 2025/0090127 A1
(43) Pub. Date: Mar. 20, 2025

(54) ELECTRIC BRUSH MODULE AND X-RAY CT APPARATUS

(71) Applicant: CANON MEDICAL SYSTEMS CORPORATION, Tochigi (JP)

(72) Inventors: Gongting TANG, Dalian (CN); Jie YAN, Dalian (CN); Qi SUN, Dalian (CN); Zehua WEN, Dalian (CN)

(73) Assignee: CANON MEDICAL SYSTEMS CORPORATION, Tochigi (JP)

(21) Appl. No.: 18/883,791

(22) Filed: Sep. 12, 2024

(30) Foreign Application Priority Data
Sep. 14, 2023 (CN) 202311189084.3

(51) Int. Cl. A61B 6/00; A61B 6/03; H01R 39/60

(52) U.S. Cl. CPC A61B 6/56; A61B 6/035; A61B 6/4435; H01R 39/60; H01R 2201/12

(57) ABSTRACT

An electric brush module according to an embodiment is used in an X-ray Computed Tomography (CT) apparatus including a slip ring and a gantry fixed part and includes an electric brush holder, an electric brush, and a wear debris collecting mechanism. The electric brush holder is provided in the gantry fixed part, has an arc shape with an opening part formed therein, and has a plate-like shape. The electric brush is attached to the electric brush holder and is configured to transmit either electric power or a signal, by being in contact with the slip ring. The wear debris collecting mechanism is attached to the electric brush holder, so as to form a substantially hermetically-closed space, together with the electric brush and the electric brush holder.

### CROSS-REFERENCE TO RELATED APPLICATIONS

This application is based upon and claims the benefit of priority from Chinese Patent Application No. 202311189084.3, filed on Sep. 14, 2023, the entire contents of all of which are incorporated herein by reference.

### FIELD

Embodiments described herein relate generally to an electric brush module and an X-ray Computed Tomography (CT) apparatus.

### BACKGROUND

At present, certain medical equipment uses a communication system in which electric power and signals are transmitted in conjunction with rotation. In such a communication system, a slip ring and an electric brush are important. The slip ring and the electric brush are used in an electric mechanical system capable of transmitting electric power and signals from a fixed part to a rotating part, in conjunction with continuously rotation. At the time of transmitting the electric power and the signals by using the slip ring and the electric brush, a brush head of the electric brush may have friction with the slip ring, and wear debris may occur in some situations.

When wear debris has occurred, there is a possibility that the wear debris may cause defects. For example, when the wear debris is scattered onto a substrate inside a gantry, there is a possibility that electric circuitry and the like may have a short circuit. As another example, when a communication system including a slip ring and an electric brush is applied to an X-ray Computed Tomography (CT) apparatus, if the wear debris is scattered inside a gantry, there is a possibility that quality of imaging may adversely be impacted. Furthermore, if the wear debris is scattered to the outside of the gantry, there is a possibility that the scattered wear debris may hit the patient, in addition to a possibility of having the gantry soiled.

For this reason, for communication systems including a slip ring and an electric brush, techniques for collecting wear debris from the electric brush have been developed, as existing techniques.

For example, in an existing communication system including a slip ring and an electric brush, a collection groove is provided underneath a slip ring shell, while the collection groove is provided with a maintenance window. With this configuration, wear debris that has occurred during operations of the slip ring and the electric brush has free fall into the collection groove provided underneath the slip ring shell. Thus, an operator is able to regularly perform cleaning, through the maintenance window.

However, in the existing communication system including the slip ring and the electric brush, the wear debris is simply let fall, by rotation gravity of a gantry, into a collection box provided in a bottom section of the slip ring, so that collection and maintenance can be performed. According to such an existing technique, although it is possible to collect some wear debris, wear debris can easily be dispersed and still gets scattered at a source where the wear debris is occurring. For this reason, the existing technique does not have a satisfactory collecting effect. Thus, there is a demand for a technique that makes it possible to collect wear debris easily and efficiently.

### BRIEF DESCRIPTION OF THE DRAWINGS

FIG. 1A is a drawing illustrating an exemplary configuration of an X-ray CT apparatus according to a first embodiment;

FIG. 1B is a schematic drawing illustrating structures of an electric brush module and the X-ray CT apparatus according to the first embodiment;

FIG. 2A is a schematic three-dimensional drawing for explaining a slip ring and the electric brush module according to the first embodiment;

FIG. 2B is an enlarged exploded three-dimensional drawing using a different observation angle from that in FIG. 2A, for explaining the electric brush module according to the first embodiment;

FIG. 3A is a three-dimensional drawing for explaining the electric brush module according to the first embodiment;

FIG. 3B is an exploded three-dimensional drawing for explaining the electric brush module according to the first embodiment;

FIG. 3C is an exploded three-dimensional drawing using a different observation angle from that in FIG. 3B, for explaining the electric brush module according to the first embodiment;

FIG. 4A is an exploded three-dimensional drawing for explaining a dust collecting mechanism in the electric brush module according to the first embodiment;

FIG. 4B is an exploded three-dimensional drawing using a different observation angle from that in FIG. 4A, for explaining the dust collecting mechanism in the electric brush module according to the first embodiment;

FIG. 5 is a three-dimensional drawing for explaining a dust collecting mechanism in an electric brush module according to a second embodiment;

FIG. 6A is an exploded three-dimensional drawing for explaining the dust collecting mechanism in the electric brush module according to the second embodiment;

FIG. 6B is an exploded three-dimensional drawing using a different observation angle from that in FIG. 6A, for explaining the dust collecting mechanism in the electric brush module according to the second embodiment;

FIG. 7 is a schematic drawing for explaining a slip ring sealing mechanism of an X-ray CT apparatus according to a third embodiment;

FIG. 8A is a three-dimensional drawing for explaining the slip ring sealing mechanism of the X-ray CT apparatus according to the third embodiment, as sectioned and observed on a cross-sectional plane perpendicular to the circumferential direction; and

FIG. 8B is a cross-sectional view taken on a cross-sectional plane perpendicular to the circumferential direction, for explaining the slip ring sealing mechanism of the X-ray CT apparatus according to the third embodiment.

### DETAILED DESCRIPTION

An electric brush module according to an embodiment of the present disclosure is used in an X-ray CT apparatus including a slip ring and a gantry fixed part and includes an electric brush holder, an electric brush, and a wear debris collecting mechanism. The electric brush holder is provided in the gantry fixed part, has an arc shape with an opening part formed therein, and has a plate-like shape. The electric brush is attached to the electric brush holder and is configured to transmit either electric power or a signal, by being in contact with the slip ring. The wear debris collecting mechanism is attached to the electric brush holder, so as to form a substantially hermetically-closed space, together with the electric brush and the electric brush holder.

An electric brush module and an X-ray CT apparatus according to an embodiment will be explained below, with reference to the accompanying drawings.

To begin with, an X-ray CT apparatus to which the electric brush module according to the present embodiment is applied will be explained.

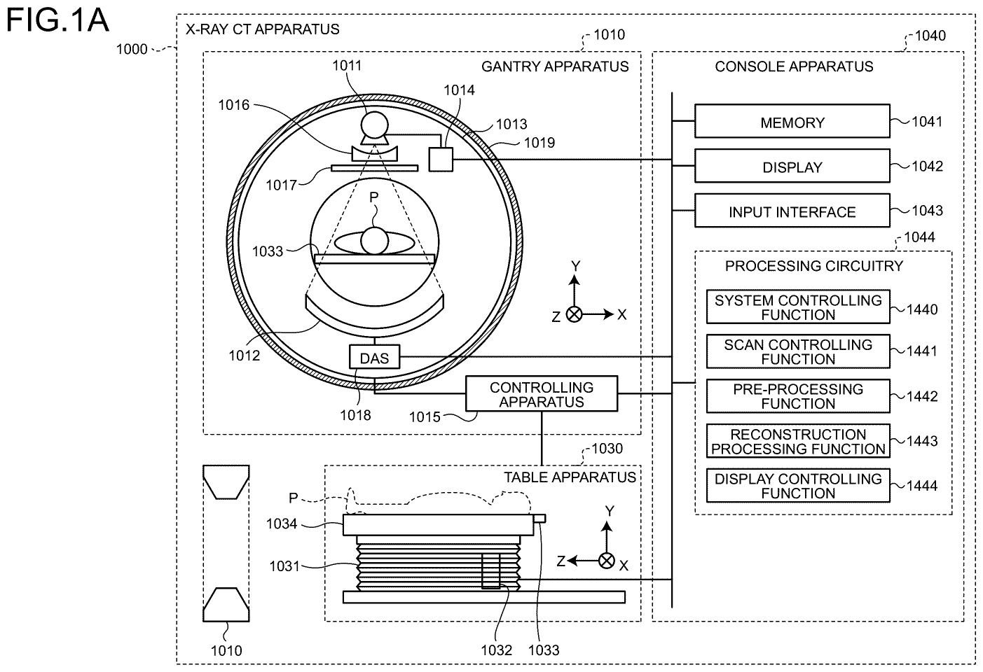

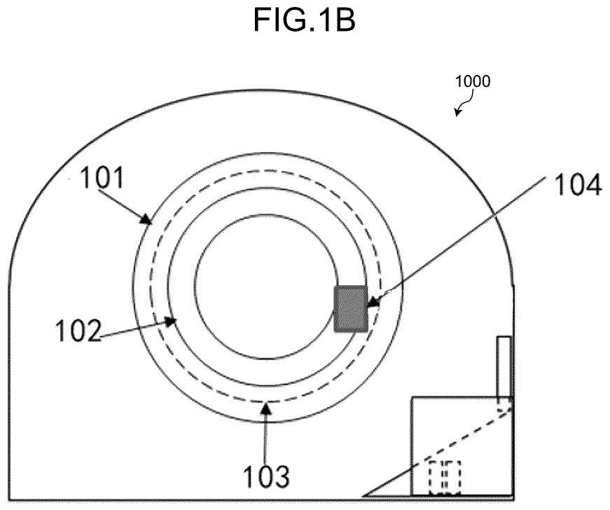

FIG. 1A is drawing illustrating an exemplary configuration of an X-ray CT apparatus 1000 according to the present embodiment.

The X-ray CT apparatus 1000 is configured to acquire CT image data of an examined subject (hereinafter, “patient”). More specifically, the X-ray CT apparatus 1000 is configured to rotationally move an X-ray tube and an X-ray detector while the patient is placed substantially at the center and configured to acquire projection data by detecting X-rays that have passed through the patient. Further, on the basis of the acquired projection data, the X-ray CT apparatus 1000 is configured to generate CT image data. As illustrated in FIG. 1A, the X-ray CT apparatus 1000 includes a gantry apparatus 1010, a table apparatus 1030, and a console apparatus 1040.

In the present embodiment, a rotation axis of a rotating frame 1013 in a non-tilt state or the longitudinal direction of a tabletop 1033 of the table apparatus 1030 will be referred to as a Z-axis direction. Further, the axial direction orthogonal to the Z-axis direction and parallel to a floor surface will be referred to as an X-axis direction. Further, the axial direction orthogonal to the Z-axis direction and perpendicular to the floor surface will be referred to as a Y-axis direction. Although FIG. 1A depicts the gantry apparatus 1010 from multiple directions for the sake of convenience in the explanation, the X-ray CT apparatus 1000 includes the single gantry apparatus 1010 in the depicted example.

The gantry apparatus 1010 includes an X-ray tube 1011, an X-ray detector 1012, the rotating frame 1013, an X-ray high-voltage apparatus 1014, a controlling apparatus 1015, a wedge 1016, a collimator 1017, a Data Acquisition System (DAS) 1018, and a fixed frame 1019.

The X-ray tube 1011 is a vacuum tube having a negative pole (a filament) configured to generate thermo electrons and a positive pole (a target or an anode) configured to generate X-rays in response to collision of thermo electrons thereon. The X-ray tube 1011 is configured to generate X-rays to be radiated onto a patient P, by causing the thermo electrons to be emitted from the negative pole toward the positive pole, with application of high voltage from the X-ray high-voltage apparatus 1014. For instance, examples of the X-ray tube 1011 include a rotating anode X-ray tube configured to generate the X-rays by having the thermo electrons emitted onto a rotating anode.

The wedge 1016 is a filter for adjusting the amount of the X-rays emitted from the X-ray tube 1011. More specifically, the wedge 1016 is a filter configured to pass and attenuate the X-rays emitted from the X-ray tube 1011, so that the X-rays emitted from the X-ray tube 1011 onto the patient P have a predetermined distribution. For example, the wedge 1016 may be a wedge filter or a bow-tie filter and is a filter obtained by processing aluminum or the like so as to have a predetermined target angle and a predetermined thickness.

The collimator 1017 is configured with lead plates or the like for narrowing down a radiation range of the X-rays that have passed through the wedge 1016 and is configured to form a slit with a combination of the plurality of lead plates or the like. The collimator 1017 may be referred to as an X-ray limiter. Further, although FIG. 1A illustrates an example in which the wedge 1016 is arranged between the X-ray tube 1011 and the collimator 1017, the collimator 1017 may be arranged between the X-ray tube 1011 and the wedge 1016. In that situation, the wedge 1016 is configured to pass and attenuate the X-rays which were emitted from the X-ray tube 1011 and of which the radiation range has been restricted by the collimator 1017.

The X-ray detector 1012 includes a plurality of detecting elements configured to detect X-rays. Each of the detecting elements included in the X-ray detector 1012 is configured to detect the X-rays that were emitted from the X-ray tube 1011 and have passed through the patient P and is configured to output a signal corresponding to a detected X-ray amount to the DAS 1018. The X-ray detector 1012 includes, for example, a plurality of arrays of detecting elements in each of which a plurality of detecting elements are arranged in a channel direction along an arc centered on a focal point of the X-ray tube 1011. For example, the X-ray detector 1012 has a structure in which the plurality of arrays of detecting elements are arranged in a row direction (a slice direction), while each array has the plurality of detecting elements arranged in the channel direction.

For example, the X-ray detector 1012 is an indirect conversion-type detector including a grid, a scintillator array, and an optical sensor array. The scintillator array includes a plurality of scintillators. Each of the scintillators includes a scintillator crystal that outputs light in a photon quantity corresponding to the amount of X-rays that have become incident thereto. The grid is arranged on a surface of the scintillator array positioned on the X-ray incident side and includes an X-ray blocking board that absorbs scattered X-rays. The grid may be referred to as a collimator (a one-dimensional collimator or a two-dimensional collimator). The optical sensor array has a function of converting the amounts of light from the scintillators into corresponding electrical signals and includes optical sensors such as photodiodes, for example. Alternatively, the X-ray detector 1012 may be a direct conversion-type detector including semiconductor elements configured to convert incident X-rays into electrical signals.

The X-ray high-voltage apparatus 1014 includes: a high-voltage generating apparatus including electric circuitry such as a transformer and a rectifier or the like and configured to generate the high voltage to be applied to the X-ray tube 1011; and an X-ray controlling apparatus configured to control output voltage corresponding to the X-rays to be generated by the X-ray tube 1011. The high-voltage generating apparatus may be of a transformer type or an inverter type. Further, the X-ray high-voltage apparatus 1014 may be provided for the rotating frame 1013 or may be provided for the fixed frame 1019. In the present example, the fixed frame 1019 is a frame configured to rotatably support the rotating frame 1013 and has a rotating mechanism for causing the rotating frame 1013 to rotate.

The DAS 1018 is configured to acquire the signals of the X-rays detected by the detecting elements included in the X-ray detector 1012. For example, while including an amplifier configured to perform an amplifying process on the electrical signals output from the detecting elements and an Analog/Digital (A/D) converter configured to convert the electrical signals into digital signals, the DAS 1018 is configured to generate detection data. The DAS 1018 is realized by using a processor, for example.

The rotating frame 1013 is an annular frame configured to support the X-ray tube 1011 and the X-ray detector 1012 so as to oppose each other and to cause the controlling apparatus 1015 to rotate the X-ray tube 1011 and the X-ray detector 1012. For example, the rotating frame 1013 is cast by using aluminum as a material thereof. In addition to the X-ray tube 1011 and the X-ray detector 1012, the rotating frame 1013 is also able to support the X-ray high-voltage apparatus 1014, the wedge 1016, the collimator 1017, the DAS 1018, and/or the like. In addition, the rotating frame 1013 is also able to support other various elements that are not illustrated in FIG. 1A.

The controlling apparatus 1015 has processing circuitry including a Central Processing Unit (CPU) or the like and a driving mechanism such as a motor and an actuator, or the like. The controlling apparatus 1015 is configured to receive an input signal from an input interface 1043 and to control operations of the gantry apparatus 1010 and the table apparatus 1030. For example, the controlling apparatus 1015 is configured to control the rotation of the rotating frame 1013, tilting of the gantry apparatus 1010, operations of the table apparatus 1030 and the tabletop 1033, and/or the like. In an example, as the control to tilt the gantry apparatus 1010, the controlling apparatus 1015 is configured to rotate the rotating frame 1013 on an axis parallel to the X-axis direction, according to inclination angle (tilt angle) information input thereto. The controlling apparatus 1015 may be provided for the gantry apparatus 1010 or may be provided for the console apparatus 1040.

The table apparatus 1030 is an apparatus on which the patient P to be imaged is placed and moved and includes a base 1031, a table driving apparatus 1032, the tabletop 1033, and a supporting frame 1034. The base 1031 is a casing configured to support the supporting frame 1034 so as to be movable in vertical directions. The table driving apparatus 1032 is a driving mechanism configured to move the tabletop 1033 on which the patient P is placed, in the long-axis directions of the tabletop 1033, and includes a motor and an actuator, or the like. The tabletop 1033 provided on the top face of the supporting frame 1034 is a board on which the patient P is placed. In addition to the tabletop 1033, the table driving apparatus 1032 may be configured to move the supporting frame 1034 in the long-axis directions of the tabletop 1033.

The console apparatus 1040 includes a memory 1041, a display 1042, the input interface 1043, and processing circuitry 1044. Although the console apparatus 1040 is described as being separate from the gantry apparatus 1010, the gantry apparatus 1010 may include the console apparatus 1040 or one or more of the constituent elements of the console apparatus 1040.

The memory 1041 is realized, for example, by using a semiconductor memory element such as a Random Access Memory (RAM) or a flash memory, or a hard disk, an optical disc, or the like. The memory 1041 is configured to store therein the projection data and the CT image data, for example. Further, for instance, the memory 1041 is configured to store therein programs used by the circuitry included in the X-ray CT apparatus 1000 for realizing the functions thereof. In another example, the memory 1041 may be realized by using a server group (a cloud) connected to the X-ray CT apparatus 1000 via a network.

The display 1042 is configured to display various types of information. For example, the display 1042 is configured to display various types of images generated by the processing circuitry 1044 and to display a Graphical User Interface (GUI) for receiving various types of operations from an operator. For example, the display 1042 may be a liquid crystal display or a Cathode Ray Tube (CRT) display. Alternatively, the display 1042 may be provided for the gantry apparatus 1010. Further, the display 1042 may be of a desktop type or may be configured by using a tablet terminal or the like capable of wirelessly communicating with the main body of the console apparatus 1040.

The input interface 1043 is configured to receive various types of input operations from the operator, to convert the received input operations into electrical signals, and to output the electrical signals to the processing circuitry 1044. For example, the input interface 1043 is configured to receive, from the operator, the input operations about a reconstruction condition used at the time of reconstructing the CT image data, an image processing condition used at the time of generating a post-processed image from the CT image data, and the like. For example, the input interface 1043 is realized by using a mouse, a keyboard, a trackball, a switch, a button, a joystick, a touchpad on which input operations can be performed by touching an operation surface thereof, a touch screen in which a display screen and a touchpad are integrally formed, contactless input circuitry using an optical sensor, audio input circuitry, and/or the like. Alternatively, the input interface 1043 may be provided for the gantry apparatus 1010. In another example, the input interface 1043 may be configured by using a tablet terminal or the like capable of wirelessly communicating with the main body of the console apparatus 1040. Further, the input interface 1043 does not necessarily need to include physical operation component parts such as the mouse, the keyboard, and/or the like. For instance, possible examples of the input interface 1043 include processing circuitry configured to receive an electrical signal corresponding to an input operation from an external input mechanism provided separately from the console apparatus 1040 and to output the electrical signal to the processing circuitry 1044.

The processing circuitry 1044 is configured to control operations of the entirety of the X-ray CT apparatus 1000. For example, the processing circuitry 1044 is configured to execute a system controlling function 1440, a scan controlling function 1441, a pre-processing function 1442, a reconstruction processing function 1443, and a display controlling function 1444.

The system controlling function 1440 is configured to control various types of functions of the processing circuitry 1044 on the basis of input operations received from the operator via the input interface 1043.

The scan controlling function 1441 is configured to perform a scan using the X-rays, on the patient P. For example, the scan controlling function 1441 is configured to control the scan, on the basis of an input operation received from the operator via the input interface 1043. More specifically, the scan controlling function 1441 is configured to control the output voltage from the high-voltage generating apparatus, by transmitting a control signal to the X-ray high-voltage apparatus 1014, on the basis of an input operation. Further, the scan controlling function 1441 is configured to control the data acquisition performed by the DAS 1018, by transmitting a control signal to the DAS 1018.

The pre-processing function 1442 is configured to generate pre-processed data, by performing pre-processing processes on the X-ray detection data transmitted from the DAS 1018. More specifically, the pre-processing function 1442 is configured to generate the pre-processed data by performing a logarithmic conversion process and/or correcting processes such as an offset correction, a sensitivity correction, and a beam hardening correction. The data (the X-ray detection data) before the pre-processing processes and the data resulting from the pre-processing processes may collectively be referred to as projection data.

The reconstruction processing function 1443 is configured to generate the CT image data by reconstructing the projection data generated by the pre-processing function 1442, while using any of various types of reconstruction methods (e.g., a back projection method such as a Filtered Backprojection (FBP) method or a successive approximation method). Further, the reconstruction processing function 1443 is configured to store the generated CT image data into the memory 1041.

The display controlling function 1444 is configured to cause the display 1042 to display various types of images generated by the processing circuitry 1044. For example, the display controlling function 1444 is configured to cause the display 1042 to display the CT image data generated by the reconstruction processing function 1443.

In the X-ray CT apparatus 1000 illustrated in FIG. 1A, the processing functions are stored in the memory 1041, in the form of computer-executable programs. The processing circuitry 1044 is represented by one or more processors configured to realize the functions corresponding to the programs by reading and executing the programs from the memory 1041. In other words, the processing circuitry 1044 that has read the programs has the functions corresponding to the read programs.

The term “processor” used in the above explanations may denote, for example, a Central Processing Unit (CPU), a Graphics Processing Unit (GPU), or circuitry such as an Application Specific Integrated Circuit (ASIC). Further, the term “processor” may denote circuitry such as a programmable logic device. Examples of the programable logic device include a Simple Programmable Logic Device (SPLD) and a Complex Programmable Logic Device (CPLD). Other examples of the programable logic device include a Field Programmable Gate Array (FPGA). When the one or more processors are each a CPU, for example, the processors are configured to realize the functions by reading and executing the programs saved in the memory 1041. In contrast, when the one or more processors are each an ASIC, for example, instead of having the programs saved in the memory 1041, the programs are directly incorporated in the circuitry of the one or more processors. Further, the processors in the present embodiments do not each necessarily have to be structured as a single piece of circuitry. It is also acceptable to structure one processor by combining together a plurality of pieces of independent circuitry so as to realize the functions thereof. Further, it is also acceptable to integrate two or more of the constituent elements depicted in FIG. 1A into one processor, so as to realize the functions thereof.

The X-ray CT apparatus 1000 illustrated in FIG. 1A has thus been explained. In the following sections, certain elements related to a technical concept of the present disclosure will be explained, while explanations about the other elements may be omitted. Further, some of the elements having the same or similar functions may be referred to by using the same reference characters, and duplicate explanations thereof may be omitted.

Further, in FIG. 1B explained later, as for a slip ring 103 having a circular shape, the direction of the diameter will be referred to as a radial direction. One of the sides approaching the center of the circle from the slip ring 103 will be referred to as a radially inner side or radially inward. One of the sides getting away from the center of the circle of the slip ring 103 will be referred to as a radially outer side or radially outward. The direction along the circumference of the slip ring 103 will be referred to as a circumferential direction.

Further, the drawings are presented for the sake of convenience in the explanations. Thus, the situations depicted in the drawings may not necessarily be situations exhibited while the elements are in use in reality. Also, for the sake of convenience in the presentation, the displayed scales of the elements in the drawings may not be completely the same as one another. Further, the presentations may not be in complete correspondence among the drawings. For the sake of convenience, one or more of the elements may be omitted from the drawings.

### First Embodiment

An electric brush module 104 according to a first embodiment will be explained, with reference to FIGS. 1B to 4B. FIG. 1B is a schematic drawing illustrating structures of the electric brush module 104 and the X-ray CT apparatus 1000 according to the first embodiment.

As illustrated in FIG. 1B, the X-ray CT apparatus 1000 according to the first embodiment includes: a rotating part 101, a fixed part (corresponding to the gantry fixed part) 102, the slip ring 103, and the electric brush module 104 provided in the fixed part 102.

The rotating part 101 illustrated in FIG. 1B corresponds to the rotating frame 1013 illustrated in FIG. 1A, whereas the fixed part 102 illustrated in FIG. 1B corresponds to the fixed frame 1019 illustrated in FIG. 1A. As explained above, the fixed part 102 is configured to support the rotating part 101, while the electric brush module 104 is attached to the fixed part 102.

The slip ring 103 is provided in the rotating part 101, is structured by using an electrically conductive material, and is used for performing electric power transmission and signal transmission between the rotating part 101 and the fixed part 102, in collaboration with the electric brush module 104.

The electric brush module 104 is provided in the fixed part 102 and is structured by using an electrically conductive material. When the X-ray CT apparatus 1000 is in operation, in conjunction with the electric brush module 104 being in contact with the slip ring 103 while the slip ring 103 is rotating, the electric power transmission and the signal transmission are performed between the rotating part 101 and the fixed part 102.

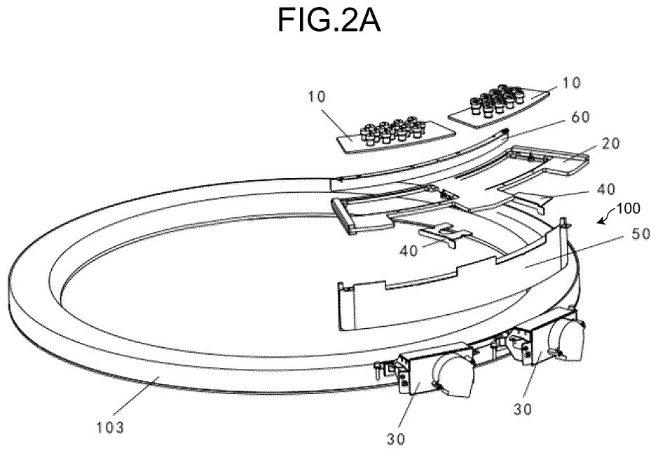

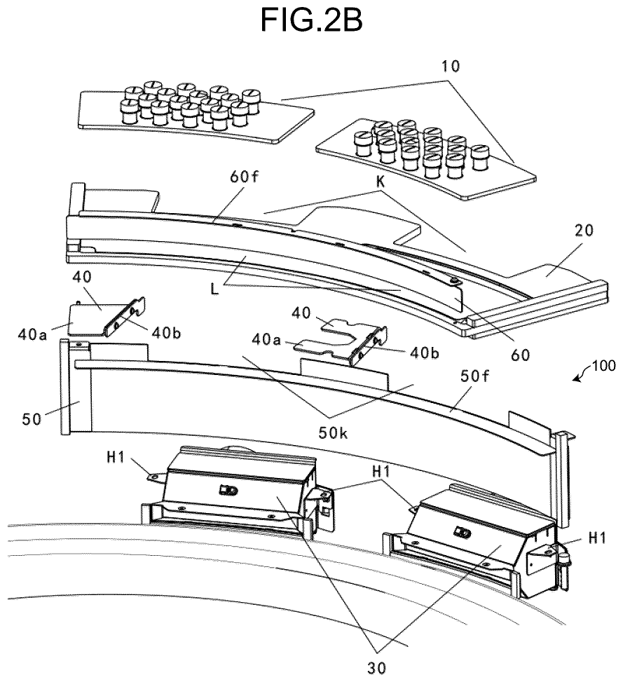

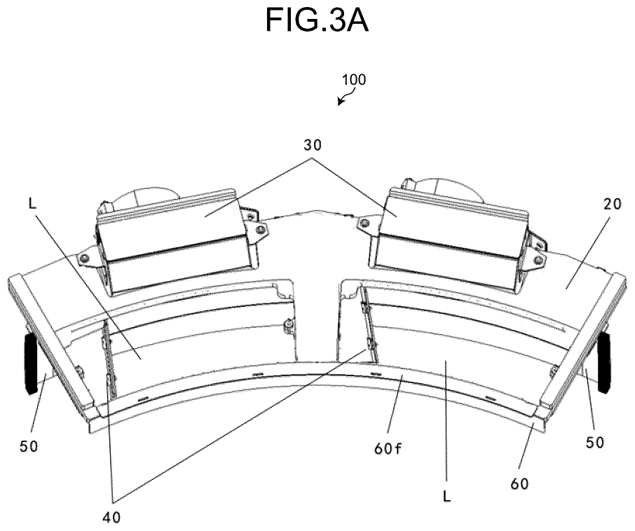

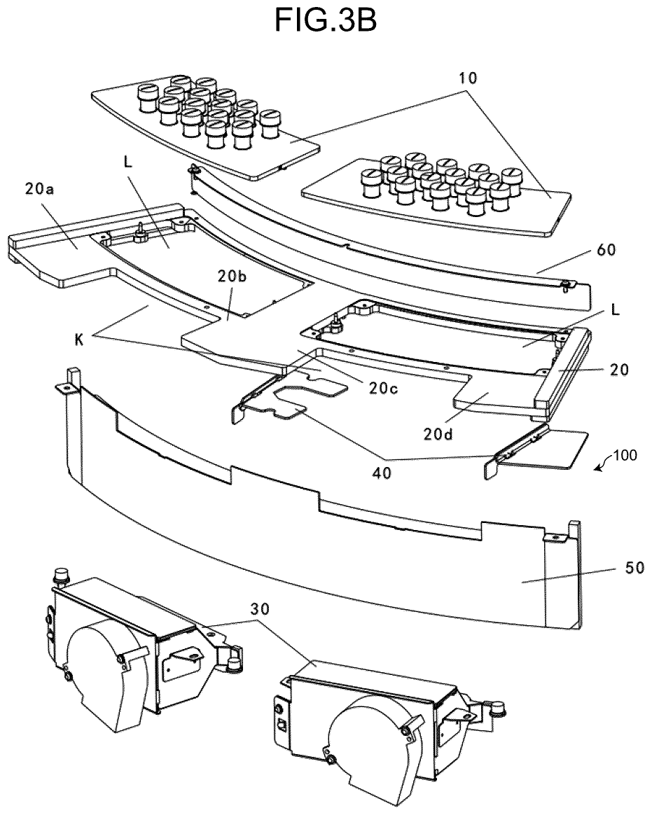

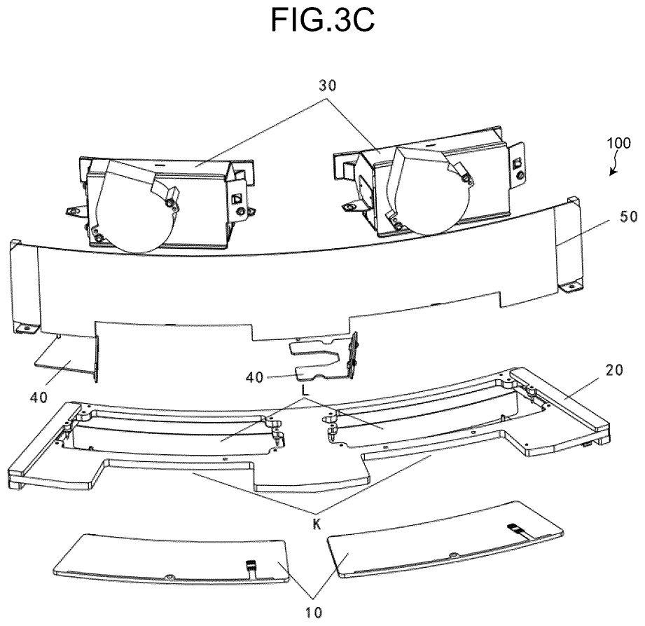

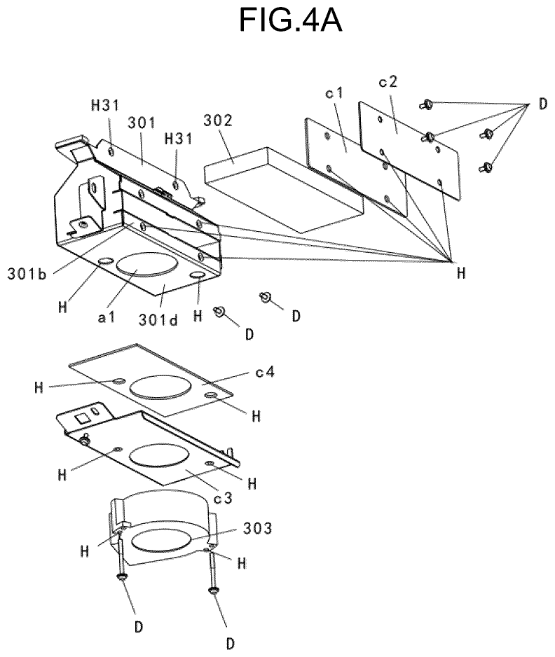

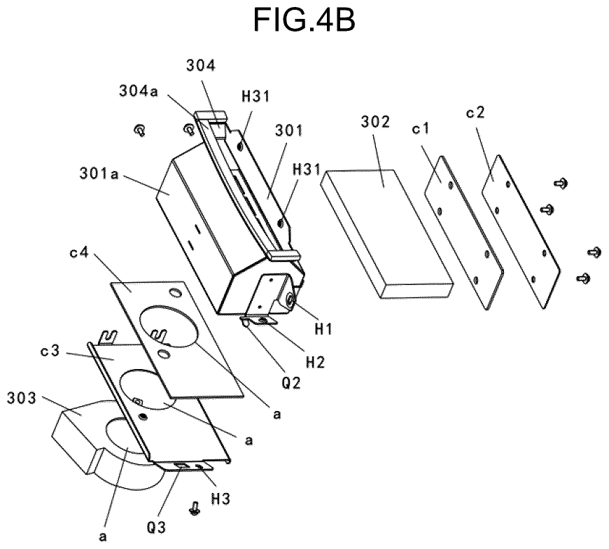

Next, the electric brush module 104 according to the first embodiment will be explained, with reference to FIGS. 2A to 4B. FIG. 2A is a schematic three-dimensional drawing for explaining the slip ring and the electric brush module 104 according to the first embodiment. FIG. 2B is an enlarged exploded three-dimensional drawing using a different observation angle from that in FIG. 2A, for explaining the electric brush module 104 according to the first embodiment. FIG. 3A is a three-dimensional drawing for explaining the electric brush module 104 according to the first embodiment. FIG. 3B is an exploded three-dimensional drawing for explaining the electric brush module 104 according to the first embodiment. FIG. 3C is an exploded three-dimensional drawing using a different observation angle from that in FIG. 3B, for explaining the electric brush module 104 according to the first embodiment. FIG. 4A is an exploded three-dimensional drawing for explaining a dust collecting mechanism 30 in the electric brush module 104 according to the first embodiment. FIG. 4B is an exploded three-dimensional drawing using a different observation angle from that in FIG. 4A, for explaining the dust collecting mechanism 30 in the electric brush module 104 according to the first embodiment.

For the sake of viewability, FIGS. 2A and 2B each depict only the slip ring 103 and the electric brush module 104, whereas FIGS. 3A to 3C each depict only the electric brush module 104, while the other elements are omitted from the drawings.

The electric brush module 104 is used in the X-ray CT apparatus 1000 including the slip ring 103 and the gantry fixed part (the fixed part 102). The electric brush module 104 includes an electric brush holder 20, electric brushes 10, and a wear debris collecting mechanism 100. The electric brushes 10 are attached to the electric brush holder 20 and are configured to transmit either electric power or a signal by being in contact with the slip ring 103. The electric brush holder 20 is provided in the fixed part 102. The electric brush holder 20 is structured to have an arc shape with an opening part formed therein and has a plate-like shape. The wear debris collecting mechanism 100 is attached to the electric brush holder 20 so as to form a substantially hermetically-closed space, together with the electric brushes 10 and the electric brush holder 20. In this situation, the “substantially hermetically-closed space” being formed by the wear debris collecting mechanism 100, the electric brushes 10, and the electric brush holder 20 denotes a state in which the slip ring 103 is covered by the wear debris collecting mechanism 100, the electric brushes 10, and the electric brush holder 20, to such an extent that it is possible to collect substantially all wear debris.

In this situation, from the top side of the drawing page of FIG. 3B, the electric brushes 10 are attached, by screws or the like for example, to opening parts L of the electric brush holder 20, in such a manner that the electric brushes 10 are in contact with the rotating slip ring 103 on the bottom side, as seen in the drawing page of FIG. 3B, of the electric brush holder 20. As a result, when seen from the bottom side of the drawing page of FIG. 3B, the electric brushes 10 are exposed from the opening parts L. While being in contact with the slip ring 103, the electric brushes 10 have friction as the slip ring 103 rotates. The wear debris caused thereby drifts from a contact part between the electric brushes 10 and the slip ring 103 along with movement of air caused by the rotation of the slip ring 103, and thus a flow of wear debris is formed. In addition, there is also wear debris that is scattered around in every direction from the contact part between the electric brushes 10 and the slip ring 103, without forming any flow of wear debris.

Further, as illustrated in FIGS. 3A to 3C, the electric brush holder 20 has formed therein: opening parts L used for attaching the electric brushes 10; and cutout parts K used for attaching the dust collecting mechanisms 30 explained later.

As illustrated in FIGS. 2A to 3C, the wear debris collecting mechanism 100 includes, for example, the dust collecting mechanisms 30, air passage partitions 40, a sealing plate 50, and a wear debris partition 60.

The dust collecting mechanisms 30 may be called dust collecting boxes and are each attached to the surface on one of the sides of the electric brush holder 20 on which the electric brushes 10 are attached, on the radially outer side of the electric brush holder 20. The dust collecting mechanisms 30 are accommodated in the cutout parts K of the electric brush holder 20. Further, it is desirable to provide the dust collecting mechanisms 30 in the vicinity of the contact part between the electric brushes 10 and the slip ring 103, i.e., in the vicinity of a source where the wear debris occurs (hereinafter, “wear debris occurrence source”). With this configuration, in the vicinity of the wear debris occurrence source, it is possible to collect, into the dust collecting mechanisms 30, the wear debris occurring at the contact part between the electric brushes 10 and the slip ring 103 due to the friction between the electric brushes 10 and the slip ring 103. In addition, it is desirable when the quantity of the dust collecting mechanisms 30 is equal to the quantity of the opening parts L formed in the electric brush holder 20, so that each of the dust collecting mechanisms 30 is provided in correspondence with a different one of the opening parts L formed in the electric brush holder 20.

As illustrated in FIGS. 4A and 4B, each of the dust collecting mechanisms 30 incudes a casing 301, adsorbent cotton 302, and an air suction mechanism 303.

As illustrated in FIG. 4B, the casing 301 is attached to the electric brush holder 20. More specifically, the electric brush holder 20 is attached to a face 301a being one of a plurality of faces structuring the casing 301. Further, formed on the inside of the casing 301 is a chamber that opens onto a face 301b opposing the face 301a of the casing 301.

Further, as illustrated in FIG. 4B, a debris entrance 304 for sucking wear debris through is formed in a surface of the casing 301 that is positioned on the radially inner side in the state where the casing 301 is attached to the electric brush holder 20. Further, a guiding part 304a is formed around the debris entrance 304.

Further, as illustrated in FIG. 4A, an opening a1 is formed in a surface 301d of the casing 301 that is positioned on the radially outer side in the state where the casing 301 is attached to the electric brush holder 20.

The adsorbent cotton 302 is detachably attached to the chamber of the casing 301. For example, the adsorbent cotton 302 may be configured by using a porous and breathable material such as sponge and is used for the purpose of adsorbing, with certainty, the wear debris collected by the dust collecting mechanism 30, so that the wear debris does not get scattered.

The air suction mechanism 303 is attached to the casing 301 so as to communicate with the opening al formed in the surface 301d of the casing 301. According to one method for attaching the air suction mechanism 303 to the casing 301, for example, the air suction mechanism 303 may be attached to the surface 301d of the casing 301, by screw holes H formed in the air suction mechanism 303, screw holes H formed in the surface 301d of the casing 301, and screws D serving as fastening members. Further, for example, the air suction mechanism 303 may be one selected from among an axial fan, a centrifugal fan, and a bladeless fan. Further, as long as it is possible to suck air out of the casing 301 and to generate negative pressure so that the wear debris is adsorbed, the air suction mechanism 303 is not limited to the fans listed above and may be configured by using other elements.

Further, each of the dust collecting mechanisms 30 includes a plurality of plate members c1 to c4. Each of the plate members c1 to c4 is formed to have substantially the same size as a corresponding one of the surfaces of the casing 301. Further, each of the plate members c1 to c4 has formed therein screw holes H corresponding to the screw holes H formed in a corresponding one of the surfaces of the casing 301.

Among the plate members, the plate members c1 and c4 are configured by using an elastically deformable material such as rubber, for example, so as to have a sealing function and to be used for the purpose of preventing the wear debris from jumping out. Further, although the drawing illustrates the example in which the dust collecting mechanism 30 includes the plate members c1 and c4, the plate members c1 and c4 are not requisite and may be omitted depending on situations.

Further, the plate member c2 is configured by using a metallic material and is used for the purpose of supporting and fastening the adsorbent cotton 302. More specifically, the plate member c2 is formed to have the same size as the face 301b of the casing 301. Also, the plate member c2 has formed therein screw holes H in one-to-one correspondence with the screw holes H formed in the face 301b of the casing 301. By using this configuration, after the adsorbent cotton 302 is placed in the chamber of the casing 301, the adsorbent cotton 302 is securely attached to the chamber of the casing 301 by fastening the plate member c2 onto the face 301b of the casing 301 while using screws or the like. Further, when the adsorbent cotton 302 requires maintenance and cleaning, it is possible to take out and clean the adsorbent cotton 302 placed in the chamber of the casing 301, by loosening the screws used for fastening the plate member c2 to the face 301b of the casing 301.

Further, when the plate member c1 is provided, the plate member c1 has formed therein screw holes H in one-to-one correspondence with the holes formed in the face 301b of the casing 301, similarly to the plate member c2. In that situation, after the adsorbent cotton 302 is placed in the chamber of the casing 301, the adsorbent cotton 302 is securely attached to the chamber of the casing 301, with the use of a sealing scheme, by fastening both of the plate members c1 and c2 to the face 301b of the casing 301 while using screws or the like.

Further, the plate member c3 is configured by using a metallic material and is used for supporting and fastening the air suction mechanism 303. More specifically, the plate member c3 is formed to have the same size as the surface 301d (the face 301d) of the casing 301. Also, the plate member c3 has formed therein screw holes H and an opening “a” in one-to-one correspondence with the screw holes H and the opening a1 formed in the face 301d of the casing 301. In this situation, in the state where a suction port of the air suction mechanism 303 is communicating with the opening al formed in the face 301d of the casing 301, the air suction mechanism 303 is securely attached to the face 301d of the casing 301 by fastening the plate member c3 to the face 301d of the casing 301 while using screws or the like.

Further, when the plate member c4 is provided, similarly to the plate member c3, the plate member c4 has formed therein holes (which may be screw holes) H and an opening “a” in one-to-one correspondence with the screw holes H formed in the face 301d of the casing 301. In this situation, in the state where the suction port of the air suction mechanism 303 is communicating with the opening al formed in the face 301d of the casing 301 and with the opening formed in the plate member c4, the air suction mechanism 303 is securely attached to the face 301d of the casing 301, with the use of a sealing scheme, by fastening both of the plate members c3 and c4 to the face 301d of the casing 301, while using screws or the like.

In addition, the plate member c3 is further provided with fastening members at two ends thereof in terms of the length direction, while each of the fastening members is provided with a hole H3 and/or a notch Q3 corresponding to a hole (e.g., H2) or a jaw Q2 provided on the casing 301. With this configuration, by performing twisting or engagement process using screws or the like, the plate member c3 is securely fastened to the casing 301.

Further, as illustrated in FIGS. 4A and 4B, in the vicinity of the debris entrance 304 of the casing 301, screw holes H31 are formed. For example, from the top side of the drawing page of FIG. 3B, screws or the like are sequentially put through the screw holes H31 in the casing 301 and the screw holes (see FIGS. 3B and 3C) formed in a section between the opening part L and the cutout part K of the electric brush holder 20. Also, from the top side of the drawing page of FIG. 3B, for example, screws or the like are sequentially put through the screw holes H1 (see FIG. 4B) in the casing 301 and an attachment part 20a (or an attachment part 20c) (see FIGS. 3B and 3C) of the electric brush holder 20. Furthermore, from the top side of the drawing page of FIG. 3B, for example, by sequentially putting screws or the like through the screw holes H1 (see FIG. 2B) in the casing 301 and an attachment part 20b (or an attachment part 20d) of the electric brush holder 20, it is possible to attach the dust collecting mechanism 30 to the electric brush holder 20.

Further, as illustrated in FIG. 3A, the air passage partitions 40 are attached to the opening parts L of the electric brush holder 20. For example, the air passage partitions 40 are provided in correspondence with the opening parts L of the electric brush holder 20. More specifically, each of the air passage partitions 40 is attached to an end section along the circumferential direction of a corresponding one of the opening parts L of the electric brush holder 20. For example, when each of the air passage partitions 40 corresponds to a different one of the opening parts L of the electric brush holder 20, it is desirable, in the example in FIGS. 3A to 3C, to attach each of the air passage partitions 40 on the downstream side of the flow of wear debris, as the end section along the circumferential direction of the opening part L of the electric brush holder 20. However, possible embodiments are not limited to this example, and it is also acceptable to attach each of the air passage partitions 40 on the upstream side of the flow of wear debris. Further, when a set of two air passage partitions 40 corresponds to each of the opening parts L of the electric brush holder 20, it is also acceptable to attach the two air passage partitions 40 to two end sections (both of the end sections) along the circumferential direction of the corresponding one of the opening parts L of the electric brush holder 20. In other words, it is desirable to attach the air passage partitions 40 on both the upstream side and the downstream side of the flow of wear debris.

Further, as illustrated in FIG. 2B and so on, the air passage partitions 40 are each formed to have an L-shaped cross-section. More specifically, each of the air passage partitions 40 is formed by bending a long metal sheet by substantially 90 degrees and performing a punching process.

Further, as illustrated in FIG. 2B and so on, each of the air passage partitions 40 includes a base part 40a and a raised part 40b. The base part 40a has formed therein one or more screw holes (not illustrated) used for attaching the air passage partition 40 to the electric brush holder 20. The height of the raised part 40b raised from the base part 40a is shorter than the thickness of the electric brush holder 20. Each of the air passage partitions 40 extends, from the surface of the electric brush holder 20, in the direction in which the electric brushes 10 are attached, along the direction perpendicular to the plate plane direction of the electric brush holder 20. For example, for each of the air passage partitions 40, the height of the raised part 40b raised from the base part 40a is arranged to be shorter than the thickness of the electric brush holder 20.

Further, as illustrated in FIGS. 3A and 3B, it is desirable to configure each of the air passage partitions 40 so as to extend along the radial direction, from a section positioned radially more inward than a radially inward section of the contact part between the electric brushes 10 and the slip ring 103, to a section positioned radially more outward than a radially outward portion of the contact part between the electric brushes 10 and the slip ring 103. Further, it is desirable to ensure that an end section of each of the air passage partitions 40 positioned on the radially outer side extends approaching an end section of the debris entrance 304 positioned on the circumferentially outer side in the corresponding dust collecting mechanism 30 described below. With this configuration, when the wear debris occurring from a wear debris occurrence source forms a flow so as to drift to the air passage partition 40, the wear debris is guided by the air passage partition 40 to the debris entrance 304 of the dust collecting mechanism 30. In other words, because a sealed space is realized by using the air passage partition 40, the flow of wear debris is guided by the air passage partition 40. As explained above, by using the air passage partitions 40, it is possible to better guide the wear debris and to prevent the wear debris from being scattered.

Further, as illustrated in FIGS. 3A and 3B, the sealing plate 50 is attached, on the surface of the electric brush holder 20 on the side opposite from the surface on the one of the sides to which the electric brushes 10 are attached, to a section of the electric brush holder 20 positioned on the radially outer side, so as to extend approaching the slip ring 103 from the radially outer side. Further, it is desirable when the plate plane of the sealing plate 50 extends along an arc of which the arc angle is equal to that of the electric brush holder 20. It is desirable when the arc length of the sealing plate 50 is longer than the arc length of the electric brush holder 20, when measured in a location of the same radius. With these configurations, as a result of the sealing plate 50 being tightly connected to a slip ring shell 2 in a third embodiment (explained later), it is possible to form a hermetically-closed space that is excellent in collecting the wear debris. Further, it is desirable to attach the sealing plate 50 to a position radially more outward than the opening parts L of the electric brush holder 20. In addition, it is desirable when a cross-section of the sealing plate 50 in the radial direction is inverted L-shaped.

Further, as illustrated in FIG. 2B, the sealing plate 50 has formed therewith a fin sheet 50f toward the inside of the arc. The fin sheet 50f contributes to the sealing of the slip ring 103 and the electric brush holder 20 and thus prevents the wear debris from being scattered and leaking. In addition, the sealing plate 50 has formed therein cutout parts 50k used for accommodating the dust collecting mechanisms 30, in the state where the electric brush module 104 is attached.

Further, as illustrated in FIGS. 3A and 3B, the wear debris partition 60 is attached to a section of the electric brush holder 20 positioned on the radially inner side, so as to oppose the sealing plate 50 while the air passage partitions 40 are interposed therebetween. Further, a cross-section of the wear debris partition 60 in the radial direction is inverted L-shaped. The wear debris partition 60 has formed therewith a fin sheet 60f toward the outside of the arc. The wear debris partition 60 is fastened to the electric brush holder 20 by using screw holes formed in the fin sheet 60f and screws, or the like.

Further, the plate plane of the wear debris partition 60 extends along an arc of which the arc degree is equal to that of the electric brush holder 20. The arc length of the wear debris partition 60 is equal to the arc length of the electric brush holder 20, when measured in a location of the same radius. Further, it is desirable to attach the wear debris partition 60 to a position radially more inward than the opening parts L of the electric brush holder 20.

Further, it is desirable to provide the wear debris partition 60 so as to extend approaching the slip ring 103 in the direction perpendicular to the plate plane direction of the electric brush holder 20. Also, it is desirable when the wear debris partition 60 is positioned apart from the slip ring 103 by 5 mm or longer, in the direction perpendicular to the surface of the electric brush holder 20.

As explained above, the electric brush module 104 according to the present embodiment includes the electric brushes 10, the electric brush holder 20, and the wear debris collecting mechanism 100 and is thereby configured to form the substantially hermetically-closed space by using the wear debris collecting mechanism 100, the electric brushes 10, and the electric brush holder 20. With this configuration, the electric brush module 104 according to the present embodiment is able to effectively prevent the wear debris from being scattered and to shorten wear debris collecting processes.

More specifically, the wear debris collecting mechanism 100 includes the dust collecting mechanisms 30, the air passage partitions 40, the sealing plate 50, and the wear debris partition 60 and is thereby configured, while the electric brush module 104 is in operation in collaboration with the slip ring 103, to form the substantially hermetically-closed space (a first space) that makes it possible to guide the flow of wear debris, by using the electric brushes 10, the electric brush holder 20, the dust collecting mechanisms 30, the air passage partitions 40, the sealing plate 50, the wear debris partition 60, and the slip ring 103. With this configuration, the electric brush module 104 according to the present embodiment is able to effectively prevent the wear debris from being scattered and to shorten wear debris collecting processes.

Further, in the electric brush module 104 according to the present embodiment, each of the dust collecting mechanisms 30 is attached to the vicinity of the contact part between the electric brushes 10 and the slip ring 103. In addition, the debris entrance 304 of each of the dust collecting mechanisms 30 faces the first space, in the vicinity of the contact part between the electric brushes 10 and the slip ring 103, which is a wear debris occurrence source. With this configuration, in the electric brush module 104, the dust collecting mechanisms 30 are positioned close to the wear debris occurrence source, and it is therefore possible to efficiently collect the wear debris before the wear debris is scattered.

Further, in the electric brush module 104 according to the present embodiment, each of the dust collecting mechanisms 30 includes the adsorbent cotton 302 being detachably attached, so that the wear debris collected by the dust collecting mechanism 30 is adsorbed. In this manner, the collected wear debris is adsorbed with certainty, while the wear debris is prevented from being scattered.

Further, in the electric brush module 104 according to the present embodiment, each of the dust collecting mechanisms 30 includes the air suction mechanism 303. By forming the negative pressure in the dust collecting mechanism 30 and the first space, as a result of the air suction of the air suction mechanism 303, the wear debris is better guided and adsorbed, so that the wear debris is adsorbed by the adsorbent cotton with certainty and efficiently.

Further, in the electric brush module 104 according to the present embodiment, by including the fin sheet 50f, the sealing plate 50 contributes to the sealing of the slip ring 103 and the electric brush holder 20 and is thus able to prevent the wear debris from being scattered and leaking.

Further, in the electric brush module 104 according to the present embodiment, it is desirable when the arc length of the sealing plate 50 is longer than the arc length of the electric brush holder 20 in an installed location. With this configuration, as a result of the sealing plate 50 being tightly connected to a slip ring shell sealing plate 3 (explained later) in the third embodiment, it is possible to form a hermetically-closed space that is excellent in collecting the wear debris.

Further, in the electric brush module 104 according to the present embodiment, the wear debris partition 60 is provided so as to extend approaching the slip ring 103 in the direction perpendicular to the plate plane direction of the electric brush holder 20. In this situation, it is desirable when the wear debris partition 60 is positioned apart from the slip ring 103 by 5 mm or longer, in the direction perpendicular to the surface of the electric brush holder 20. With this configuration, in the electric brush module 104 according to the present embodiment, as a result of the wear debris collecting mechanism 100, the electric brushes 10, and the electric brush holder 20 forming the substantially hermetically-closed space, it is possible to prevent the wear debris from being scattered and to also shorten wear debris collecting processes. Further, in the electric brush module 104 according to the present embodiment, because the wear debris partition 60 is positioned apart from the slip ring 103, it is possible to avoid defects that may be caused by contact between the wear debris partition 60 and the slip ring 103.

Furthermore, the electric brush module 104 according to the present embodiment is provided with the two dust collecting mechanisms 30, while the two dust collecting mechanisms 30 are positioned close to each other and close to the wear debris occurrence source. Accordingly, the dust collecting mechanism 30 positioned on the downstream side of the flow of wear debris has a function of supplementing the collection of the dust collecting mechanism 30 positioned on the upstream side, where the dust collecting mechanism 30 positioned on the downstream side is able to supplementarily collect wear debris that was not collected by the dust collecting mechanism 30 positioned on the upstream side. In this manner, in the electric brush module 104 according to the present embodiment, because the wear debris is collected by the dust collecting mechanisms 30 in the positions close to the wear debris occurrence source, it is possible to decrease the scattering of the wear debris, to improve effects of the wear debris collection, and to enhance efficiency of the wear debris collection.

In addition, when the electric brush module 104 according to the present embodiment is applied to the X-ray CT apparatus 1000, because the wear debris is prevented from being scattered inside the gantry, it is possible to reduce risks for having a short circuit in the electric circuitry or the like and to also enhance quality of the imaging. In addition, when the electric brush module 104 according to the present embodiment is used, because the wear debris is also prevented from being scattered to the outside of the gantry, it is possible to reduce the risks for having patients hit by wear debris or having the gantry soiled.

### Second Embodiment

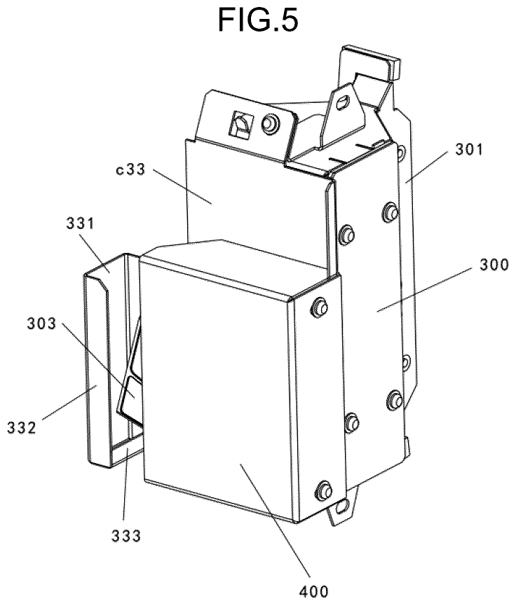

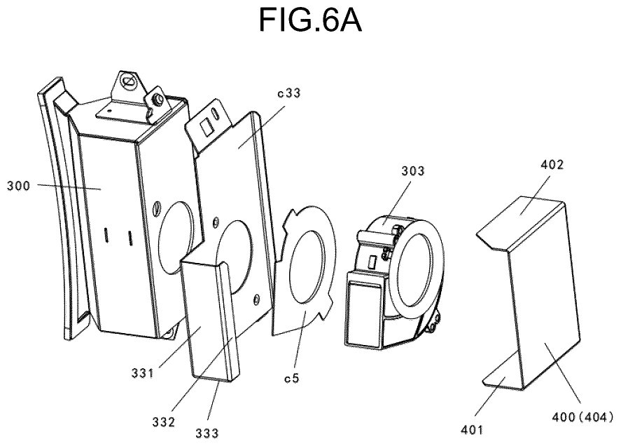

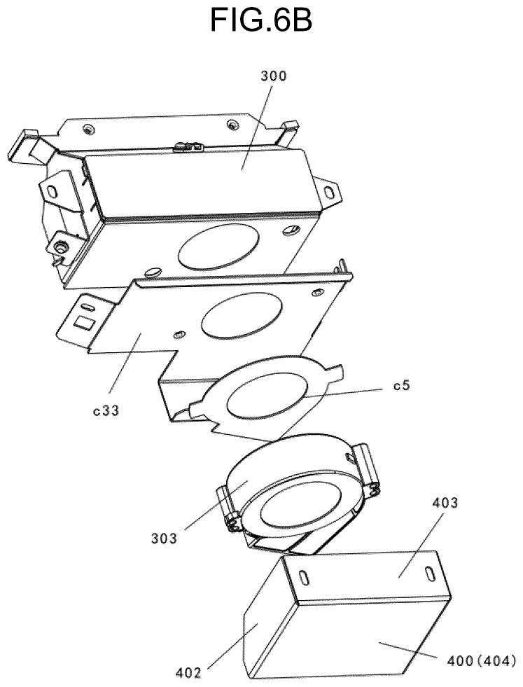

Next, the electric brush module 104 according to a second embodiment will be explained, with reference to FIGS. 5 to 6B. FIG. 5 is a three-dimensional drawing for explaining a dust collecting mechanism 300 in the electric brush module 104 according to the second embodiment. FIG. 6A is an exploded three-dimensional drawing for explaining the dust collecting mechanism 300 in the electric brush module 104 according to the second embodiment. FIG. 6B is an exploded three-dimensional drawing using a different observation angle from that in FIG. 6A, for explaining the dust collecting mechanism 300 in the electric brush module 104 according to the second embodiment.

Further, as the second embodiment is compared with the first embodiment, differences lie only in a structure related to the air suction mechanisms 303 of the dust collecting mechanisms 30, while the other features are identical to those in the first embodiment. Thus, in the present embodiment, while descriptions of some of the elements that are the same as those in the first embodiment will be omitted, only the structure related to the air suction mechanisms 303 of the dust collecting mechanisms 30 will be explained.

As illustrated in FIG. 5, the electric brush module 104 according to the second embodiment further includes soundproof mechanisms 400 and plate members c33. More specifically, a differences between the first embodiment and the second embodiment lies in that, in the second embodiment, the electric brush module 104 is provided with the plate members c33 in place of the plate members c3 of the dust collecting mechanisms 30, and in addition, the electric brush module 104 includes the soundproof mechanisms 400.

Each of the soundproof mechanisms 400 is provided for a corresponding one of the casings 301, so as to surround the air suction mechanism 303 while keeping an exhaust port exposed.

As illustrated in FIGS. 6A and 6B, the soundproof mechanism 400 is structured as a hexahedron of which two-adjacently positioned faces are open. For example, the soundproof mechanism 400 may be formed by clipping, folding, and pressing a metal sheet. More specifically, the soundproof mechanism 400 includes: a first lateral wall 401, a second lateral wall 402 opposing the first lateral wall, a third lateral wall 403 positioned between the first lateral wall 401 and the second lateral wall 402, and a bottom wall 404 positioned adjacent to the first lateral wall 401, the second lateral wall 402, and the third lateral wall 403. The soundproof mechanism 400 is sized to be capable of accommodating the air suction mechanism 303 therein. In other words, the size of the bottom wall 404 positioned adjacent to the first lateral wall 401, the second lateral wall 402, and the third lateral wall 403 is designed to match the size of the air suction mechanism 303.

The third lateral wall 403 has formed therein screw holes used for being fastened to the second lateral wall 402, by screws.

The soundproof mechanism 400 is fastened to the plate member c33 by welding, crimping, or the like.

By providing the electric brush module 104 according to the second embodiment with the soundproof mechanisms 400, noise occurring from the air suction mechanisms 303 is blocked and absorbed, so as to reduce discomfort of patients and operators. It is therefore possible to contribute to constructing an excellent medical environment and enhancing performance of the electric brush module 104 and equipment employing the electric brush module 104.

In relation to the above, for example, it is desirable to attach a noise cancelling mechanism such as sound absorbing cotton to the bottom wall 404 of the soundproof mechanism 400. With this configuration, in the electric brush module 104 according to the second embodiment, it is possible to further absorb the noise occurring at the air suction mechanism 303 and to achieve a more excellent noise prevention effect.

In addition, it is also acceptable to provide a noise cancelling mechanism such as sound absorbing cotton on one or more inner walls of the soundproof mechanism 400. In that situation also, it is possible to achieve a more excellent noise prevention effect.

Further, the soundproof mechanism 400 is configured to surround the air suction mechanism 303 while keeping the exhaust port of the air suction mechanism 303 exposed. This configuration guarantees an air discharging function of the air suction mechanism 303, to ensure that the air suction mechanism 303 is able to operate normally.

Further, in the electric brush module 104 according to the present embodiment, it is desirable to configure the dust collecting mechanism 300 so that a plate member c5 is interposed between the plate member c33 and the air suction mechanism 303. The plate member c5 may be configured by using an elastically deformable material such as rubber, for example, and has a function of absorbing noise. It is therefore possible to further cancel noise and to prevent the wear debris from jumping out more effectively.

Further, in the electric brush module 104 according to the present embodiment, it is desirable to configure the plate member c33 of the dust collecting mechanism 300 to serve as a wear debris blocking shield so as to block the exhaust port of the air suction mechanism 303, while being bent around and extending from a separator between the casing 301 and the air suction mechanism 303, in the direction toward the position of the exhaust port of the air suction mechanism 303.

More specifically, as illustrated in FIGS. 5 and 6A, the plate member c33 of the dust collecting mechanism 300 includes, in addition to the separator section positioned between the casing 301 and the air suction mechanism 303 while in an attachment state, a first extension part 331, a second extension part 332, and a third extension part 333.

The first extension part 331 extends substantially orthogonally up to a prescribed height, from at least a part (a part overlapping with the air suction mechanism) of the separator section between the casing 301 and the air suction mechanism 303, in the direction toward the position of the air suction mechanism 303. The prescribed height is slightly taller than the height of the air suction mechanism 303.

The second extension part 332 extends substantially orthogonally from the long side of the first extension part 331 in a direction approaching the air suction mechanism 303. The second extension part 332 is positioned substantially parallel to the separator section between the casing 301 and the air suction mechanism 303.

The third extension part 333 extends substantially orthogonally from one of the sides of the short side of the first extension part 331, in a direction approaching the air suction mechanism 303. While being positioned adjacent to the first extension part 331 and the second extension part 332, the third extension part 333 is configured so that the separator section between the casing 301 and the air suction mechanism 303 is positioned parallel to the separator section between the casing 301 and the air suction mechanism 303.

The first extension part 331, the second extension part 332, and the third extension part 333 are sized so as to match the size of the air suction mechanism 303.

It is possible to configure the plate member c33 of the dust collecting mechanism 300 as the wear debris blocking shield including the first extension part 331, the second extension part 332, and the third extension part 333. With this configuration, the electric brush module 104 according to the second embodiment is able to catch a small amount of wear debris jumping out of the exhaust port of the air suction mechanism 303 and to thus collect wear debris with higher certainty.

Further, it is desirable to form a wear debris collection groove on the inside of the second extension part 332. With this configuration, the electric brush module 104 according to the second embodiment is able to collect wear debris with higher certainty and is thus able to prevent the wear debris from being further scattered.

As explained above, in addition to achieving the advantageous effects of the first embodiment, the electric brush module 104 according to the second embodiment includes the soundproof mechanisms 400 and is thereby able to absorb the noise occurring from the air suction mechanisms 303 and to more effectively prevent the wear debris from jumping out. Further, because the electric brush module 104 according to the second embodiment includes the wear debris blocking shield (the plate member c33) including the first extension part 331, the second extension part 332, and the third extension part 333, it is possible to catch a small amount of wear debris jumping out of the exhaust port of the air suction mechanism 303 and to thus collect wear debris with higher certainty.

### Third Embodiment

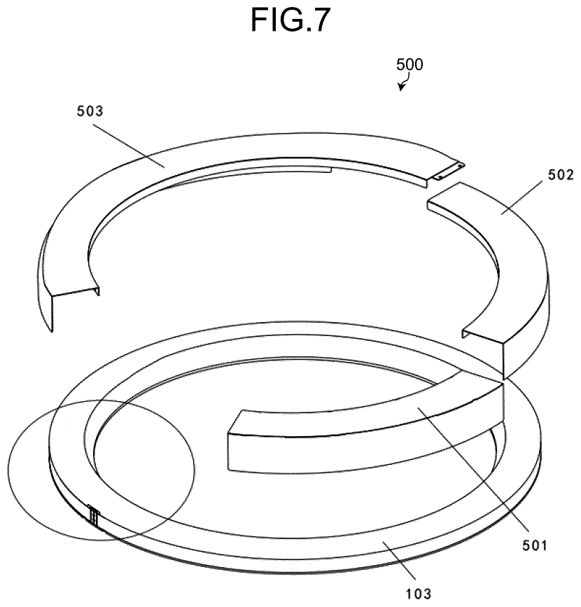

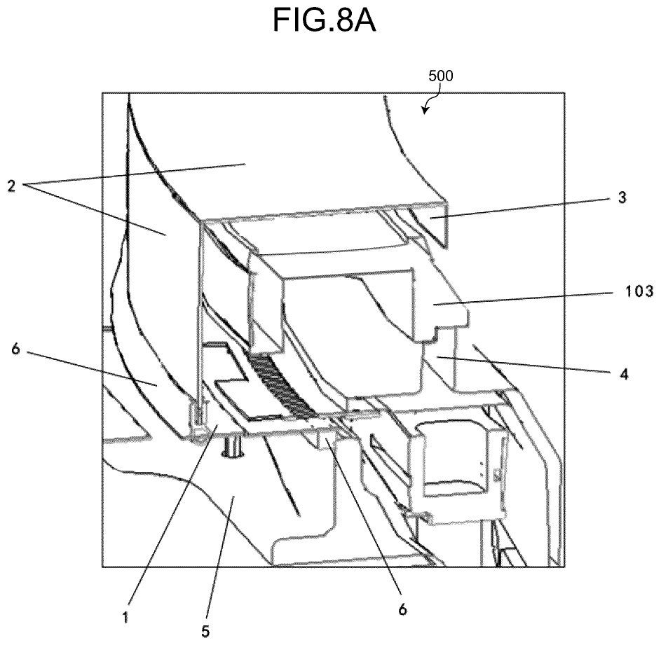

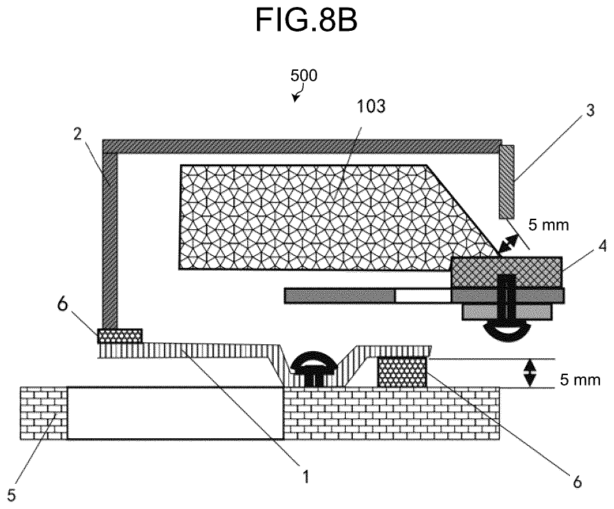

Next, the third embodiment will be explained with reference to FIGS. 7 to 8B. FIG. 7 is a schematic drawing for explaining a slip ring sealing mechanism 500 of the X-ray CT apparatus 1000 according to the third embodiment. FIG. 8A is a three-dimensional drawing for explaining the slip ring sealing mechanism 500 of the X-ray CT apparatus 1000 according to the third embodiment, as sectioned and observed on a cross-sectional plane perpendicular to the circumferential direction. FIG. 8B is a cross-sectional view taken on a cross-sectional plane perpendicular to the circumferential direction, for explaining the slip ring sealing mechanism 500 of the X-ray CT apparatus 1000 according to the third embodiment.

In this situation, in addition to the electric brush module 104 according to the first or the second embodiment, the X-ray CT apparatus 1000 according to the third embodiment further includes the slip ring sealing mechanism 500 provided for a section of the slip ring 103 other than the section thereof overlapping with the electric brush module 104. The slip ring sealing mechanism 500 will primarily be explained below. Because the structure of the electric brush module 104 included in the X-ray CT apparatus 1000 according to the third embodiment is the same as the structure of the electric brush module 104 in the first or the second embodiment, detailed explanations thereof will be omitted.

As illustrated in FIG. 7, the X-ray CT apparatus 1000 according to the present embodiment further includes the slip ring sealing mechanism 500. The slip ring sealing mechanism 500 includes a first part 501, a second part 502, and a third member 503.

The first part 501, the second part 502, and the third member 503 serving as the slip ring sealing mechanism 500 are arranged along the circumferential direction of the slip ring 103. More specifically, the first part 501, the second part 502, and the third member 503 are not provided over the entire circumferential direction of the slip ring 103, but are provided in a section other than the section (circled in FIG. 7) of the slip ring 103 overlapping, in the circumferential direction, with the electric brush module 104.

FIGS. 8A and 8B each depict a structure of the first part 501, as observed on a cross-sectional plane perpendicular to the circumferential direction. Because the structures of the first part 501, the second part 502, and the third member 503 are substantially the same as one another, detailed explanations of the structures of the second part 502 and the third member 503 as observed on a cross-sectional plane perpendicular to the circumferential direction will be omitted.

As illustrated in FIGS. 8A and 8B, the slip ring sealing mechanism 500 is configured to surround the slip ring 103 supported by a slip ring fixation ring 4, so that the slip ring 103 is substantially sealed.

The slip ring sealing mechanism 500 includes a hermetically-closing fixed plate 1, the slip ring shell 2, and the slip ring shell sealing plate 3.

The hermetically-closing fixed plate 1 is attached to a main frame 5 serving as a gantry fixed part, while being positioned between the slip ring 103 and the main frame 5. The hermetically-closing fixed plate 1 is fastened to the main frame 5 by a screw or the like.

The slip ring shell 2 is attached to the main frame 5 so as to surround the radially outer side of the slip ring 103 and the side of the slip ring 103 opposite from one of the sides opposing the main frame 5. More specifically, as illustrated in FIG. 8B, the slip ring shell 2 is attached to the hermetically-closing fixed plate 1, so as to surround the radially outer side of the slip ring 103 and the side of the slip ring 103 opposite from the one of the sides opposing the main frame 5. For example, the slip ring shell 2 may be attached to the hermetically-closing fixed plate 1 by being pasted thereon. When observed on a cross-sectional plane perpendicular to the circumferential direction, the slip ring shell 2 is inverted L-shaped. Alternatively, the slip ring shell 2 may be an integrally-formed element.

The slip ring shell sealing plate 3 is attached to the slip ring shell 2 so as to cover the radially inner side of the slip ring 103. The slip ring shell sealing plate 3 may be configured by using the same material as that of the slip ring shell 2. The slip ring shell sealing plate 3 is attached to the slip ring shell 2 by adhesion, riveting, welding, screwing, or the like. Also, the slip ring shell sealing plate 3 is tightly connected to the sealing plate 50 of the electric brush module 104 described above.

For example, as explained above, in the electric brush module 104 illustrated in FIGS. 3A and 3B, the wear debris partition 60 is positioned apart from the slip ring 103 by 5 mm or longer, in the direction perpendicular to the surface of the electric brush holder 20. Similarly, as illustrated on the right side of FIG. 8B, there is a distance of 5 mm or longer between the slip ring shell sealing plate 3 and a rim of the slip ring 103 on the radially inner side of the surface positioned on the one of the sides opposing the main frame 5.

With this configuration, the X-ray CT apparatus 1000 according to the third embodiment has a space (a second space) formed therein, by substantially sealing and surrounding the slip ring 103, while using the hermetically-closing fixed plate 1, the slip ring shell 2, and the slip ring shell sealing plate 3. It is therefore possible to guide the wear debris so as to drift through the second space and to thus efficiently prevent the wear debris from being scattered.

Further, as observed on a cross-sectional plane perpendicular to the circumferential direction, it is desirable to have the slip ring shell sealing plate 3 and the slip ring shell 2 integrally formed in a U-shape. With this configuration, for the X-ray CT apparatus 1000 according to the third embodiment, it is possible to conveniently process the component parts thereof and to enhance sealing effects.

Further, it is desirable to attach the hermetically-closing fixed plate 1 to the main frame 5, in such a manner that at least a part of the hermetically-closing fixed plate 1 has a gap from the main frame 5. In that situation, it is desirable when the gap between at least a part of the hermetically-closing fixed plate 1 and the main frame 5 is 5 mm or longer. With this configuration, in the X-ray CT apparatus 1000 according to the third embodiment, it is possible to prevent occurrence of unwanted rattling (e.g., kada-kada) noise caused by interference between the hermetically-closing fixed plate 1 and the main frame 5.

Further, it is desirable to provide: a sealing gasket 6 interposed between at least a part (the right side in FIG. 8B) of the hermetically-closing fixed plate 1 and the main frame 5; and another sealing gasket 6 interposed between at least another part (the left side in FIG. 8B) of the hermetically-closing fixed plate 1 and the slip ring shell 2. In this situation, the sealing gaskets 6 may be configured by using an elastically deformable material such as rubber, for example. The sealing gaskets 6 are each provided while being sandwiched so as to be compressed and elastically deformed. With this configuration, by using the X-ray CT apparatus 1000 according to the third embodiment, it is possible to enhance sealing effects and to prevent the occurrence of unwanted rattling (e.g., kada-kada) noise caused by contact of the component parts.

As explained above, in the X-ray CT apparatus 1000 according to the present embodiment, over the entire circumferential direction of the slip ring 103, the electric brush module 104 as well as the first part 501, the second part 502, and the third member 503 serving as the slip ring sealing mechanism 500 are provided over the entire circumferential direction of the slip ring 103. In addition, in the position where the electric brush module 104 is provided, the first space that is substantially hermetically-closed and makes it possible to guide the flow of wear debris is formed by the electric brushes, the electric brush holder 20, the dust collecting mechanisms 30, the air passage partitions 40, the sealing plate 50, the wear debris partition 60, and the slip ring 103. Further, the substantially hermetically-closed second space is formed with the first part 501, the second part 502, and the third member 503 serving as the slip ring sealing mechanism 500. In addition, the sealing plate 50 of the electric brush module 104 is tightly connected to the slip ring shell 2, while the wear debris partition 60 is tightly connected to the slip ring shell sealing plate 3. With these configurations, in the X-ray CT apparatus 1000 according to the third embodiment, the hermetically-closed spaces that are excellent in collecting the wear debris are formed over the entire circumference of the slip ring 103. It is therefore possible to effectively prevent the wear debris from being scattered and to shorten wear debris collecting processes.

Further, in the X-ray CT apparatus 1000 according to the third embodiment, because the hermetically-closed spaces that are excellent in collecting the wear debris are formed over the entire circumference of the slip ring 103, negative pressure occurs in the spaces while the air suction mechanisms 303 are in operation. It is therefore possible to efficiently guide and adsorb the wear debris and to collect the wear debris into the dust collecting mechanisms 30.

### Modification Examples

Further, in the above embodiments, the example was explained in which the electric brush holder 20 has formed therein the two opening parts L and the two cutout parts K; however possible embodiments are not limited to this example. For example, depending on situations, the electric brush holder 20 may have a single opening part L and a single cutout part K formed therein and may have three or more opening parts L and three or more cutout parts K formed therein. It is satisfactory as long as the quantities of the opening parts L and the cutout parts K correspond to the quantity of the electric brushes 10 and to the quantity of the dust collecting mechanisms 30.

Further, in the above embodiments, the example was explained in which the slip ring sealing mechanism 500 includes the three component parts, namely, the first part 501, the second part 502, and the third member 503; however, possible embodiments are not limited to this example. For instance, it is acceptable to change the component parts and the quantity of the component parts depending on actual situations, how technology is developing, specification requirements of products, and the like.

Further, in the second embodiment described above, the example was explained in which the soundproof mechanisms 400 and the wear debris blocking shields (the plate members c33) are provided at the same time; however, possible embodiments are not limited to this example. The electric brush module 104 may include one selected from between the soundproof mechanisms 400 and the wear debris blocking shields (the plate members c33).

According to at least one aspect of the embodiments described above, it is possible to efficiently collect wear debris.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An electric brush module used in an X-ray Computed Tomography (CT) apparatus including a slip ring and a gantry fixed part, the electric brush module comprising:
an electric brush holder that is provided in the gantry fixed part, has an arc shape with an opening part formed therein, and has a plate-like shape;
an electric brush attached to the electric brush holder and configured to transmit either electric power or a signal, by being in contact with the slip ring; and
a wear debris collecting mechanism attached to the electric brush holder, so as to form a substantially hermetically-closed space, together with the electric brush and the electric brush holder.

2. The electric brush module according to claim 1, wherein
the wear debris collecting mechanism includes:
a dust collecting mechanism attached, on a radially outer side of the electric brush holder, to a surface on one of sides of the electric brush holder on which the electric brush is attached, so as to be positioned in a vicinity of a contact part between the electric brush and the slip ring;
an air passage partition attached to an end section along a circumferential direction of the opening part of the electric brush holder;
a sealing plate attached, on a surface of the electric brush holder on a side opposite from the one of the sides on which the electric brush is attached, to a section of the electric brush holder positioned on the radially outer side, so as to extend approaching the slip ring from the radially outer side; and
a wear debris partition attached to a section of the electric brush holder positioned on a radially inner side, so as to oppose the sealing plate while the air passage partition is interposed therebetween.

3. The electric brush module according to claim 2, wherein
the dust collecting mechanism includes:
a casing of which one face is attached to the electric brush holder and which has formed, on an inside thereof, a chamber that opens onto a face opposing the face attached to the electric brush holder, the casing having a debris entrance for sucking wear debris formed on a surface positioned on the radially inner side in a state where the dust collecting mechanism is attached to the electric brush holder, and the casing having an opening formed on a surface positioned on the radially outer side in a state where the dust collecting mechanism is attached to the electric brush holder;
an adsorbent cotton detachably attached to the chamber of the casing; and
an air suction mechanism attached to the casing so as to communicate with the opening.

4. The electric brush module according to claim 3, wherein
the air passage partition is formed to have an L-shaped cross-section and extends along a radial direction, from a section positioned radially more inward than a radially inward section of a contact part between the electric brush and the slip ring, to a section positioned radially more outward than a radially outward portion of the contact part between the electric brush and the slip ring,
an end section of the air passage partition positioned on the radially outer side extends approaching an end section of the debris entrance positioned on a circumferentially outer side, and
the air passage partition extends, from a surface of the electric brush holder, in a direction in which the electric brush is attached, along a direction perpendicular to a plate plane direction of the electric brush holder.

5. The electric brush module according to claim 2, wherein
a plate plane of the sealing plate extends along an arc of which an arc degree is equal to that of the electric brush holder,
an arc length of the sealing plate is longer than an arc length of the electric brush holder when measured in a location of a same radius, and
the sealing plate is attached to a position more radially outward than the opening part of the electric brush holder.

6. The electric brush module according to claim 2, wherein
a cross-section of the sealing plate in a radial direction is inverted L-shaped,
a fin sheet is formed on the sealing plate toward an inside of the arc, and
the sealing plate has formed therein a cutout part for accommodating the dust collecting mechanism.

7. The electric brush module according to claim 2, wherein
a plate plane of the wear debris partition extends along an arc of which an arc degree is equal to that of the electric brush holder,
an arc length of the wear debris partition is equal to an arc length of the electric brush holder when measured in a location of a same radius, and
the wear debris partition is attached to a position radially more inward than the opening part of the electric brush holder.

8. The electric brush module according to claim 2, wherein
a cross-section of the wear debris partition in a radial direction is inverted L-shaped, and
a fin sheet is formed on the wear debris partition toward an outside of the arc.

9. The electric brush module according to claim 2, wherein
the wear debris partition is provided so as to extend approaching the slip ring in a direction perpendicular to a plate plane direction of the electric brush holder, and
the wear debris partition is positioned apart from the slip ring by 5 mm or longer, in a direction perpendicular to the surface of the electric brush holder.

10. The electric brush module according to claim 3, wherein the air suction mechanism is one selected from among an axial fan, a centrifugal fan, and a bladeless fan.

11. The electric brush module according to claim 3, further comprising: a soundproof mechanism provided for the casing so as to surround the air suction mechanism, while keeping an exhaust port exposed.

12. The electric brush module according to claim 11, wherein the soundproof mechanism is formed as a hexahedron of which two adjacently-positioned faces are open.

13. The electric brush module according to claim 3, further comprising: a wear debris blocking shield extending from a separator positioned between the casing and the air suction mechanism, in a direction toward where an exhaust port of the air suction mechanism is positioned, so as to block the exhaust port of the air suction mechanism.

14. An X-ray CT apparatus comprising:
a gantry fixed part;
a slip ring; and
an electric brush module, wherein
the electric brush modules includes
an electric brush holder that is provided in the gantry fixed part, has an arc shape with an opening part formed therein, and has a plate-like shape;
an electric brush attached to the electric brush holder and configured to transmit either electric power or a signal, by being contact in the slip ring; and
a wear debris collecting mechanism attached to the electric brush holder, so as to form a substantially hermetically-closed space, together with the electric brush and the electric brush holder.

15. The X-ray CT apparatus according to claim 14, further comprising: a slip ring sealing mechanism provided along the slip ring, for a section of the slip ring other than a section overlapping with the electric brush module, the slip ring sealing mechanism surrounding the slip ring so as to substantially seal the slip ring.

16. The X-ray CT apparatus according to claim 15, wherein
the slip ring sealing mechanism includes:
a slip ring shell attached to the gantry fixed part so as to surround a radially outer side of the slip ring and a side of the slip ring opposite from one of sides opposing the gantry fixed part; and
a slip ring shell sealing plate attached to the slip ring shell so as to cover a radially inner side of the slip ring.

17. The X-ray CT apparatus according to claim 16, wherein
the slip ring sealing mechanism further comprises a hermetically-closing fixed plate attached to the gantry fixed part while being positioned between the slip ring and the gantry fixed part, and
the slip ring shell is attached to the hermetically-closing fixed plate so as to surround the radially outer side of the slip ring and the side of the slip ring opposite from the one of the sides opposing the gantry fixed part.

18. The X-ray CT apparatus according to claim 17, wherein the hermetically-closing fixed plate is attached to the gantry fixed part, in such a manner that at least a part thereof has a gap from the gantry fixed part.

19. The X-ray CT apparatus according to claim 16, wherein there is a distance of 5 mm or longer between the slip ring shell sealing plate and a rim of the slip ring on the radially inner side of a surface positioned on the one of the sides opposing the gantry fixed part.

20. The X-ray CT apparatus according to claim 18, wherein the gap between said at least a part of the hermetically-closing fixed plate and the gantry fixed part is 5 mm or larger.
